(19) United States
(12) Patent Application Publication
(10) Pub. No.: US 2024/0194296 A1
(43) Pub. Date: Jun. 13, 2024

(54) Method for the Compression of Genome Sequence Data

(71) Applicant: ENANCIO, CESSON-SEVIGNE (FR)

(72) Inventor: Guillaume Alexandre Pascal Rizk, Rennes (FR)

(21) Appl. No.: 18/540,814

(22) Filed: Dec. 14, 2023

Related U.S. Application Data
(63) Continuation of application No. 16/567,211, filed on Sep. 11, 2019, now abandoned.

(51) Int. Cl. G16B 30/10; G06F 16/23; G16B 20/20; G16B 30/20; G16B 45/00; G16B 50/50

(52) U.S. Cl. CPC G16B 30/10; G06F 16/2365; G16B 20/20; G16B 30/20; G16B 45/00; G16B 50/50

(57) ABSTRACT

The invention relates to a reference-based method for the compression of genome sequence data produced by a sequencing machine. The sequences of nucleotides or bases, that have been previously aligned to a reference sequence, are determined to be perfectly mapped, imperfectly mapped or unmapped with the reference sequence; and then coded according to said determination. The determining step comprises comparing, for each imperfectly mapped sequence, the number of mismatches between said sequence and the reference sequence with a reference threshold value, and encoding the imperfectly mapped sequences according to distinct encoding processes, depending on the result of said comparison method for the compression of genome sequence data produced by a sequencing machine

### TECHNICAL FIELD

The field relates generally to the methods of representation of genome sequencing data produced by a sequencing machine, and more particularly to the computer-implemented methods for the compression of such genome sequencing data. This disclosure provides a reference-based compression method which allows fast compression and decompression while causing no loss of information, and which has a high compression ratio.

### BACKGROUND

Next generation sequencing machines now produce huge amounts of sequencing data at an affordable price. Recent systems produce in a single run of 36 h more than 6 billion 150-nucleotide long sequences, enough for the sequencing of 20 whole human genomes. This opens many new perspectives for the diagnostic of genetic diseases and for the development of personalized medicine, aiming to adapt treatment based on people genomic specificities.

However, this also comes with new challenges, in particular the cost related to the storage of huge amounts of data. The most used file format for raw (unaligned) sequence data is the FASTQ format, holding sequence data (string of A, C, T, G nucleotides, also called read), quality values (probabilities that the sequencing platform made a sequencing error for each nucleotide) and sequence names. This is a plain ASCII text file, usually compressed with the general purpose text compression scheme LZ (Lempel-Ziv scheme, implemented in the gzip software). However, the use of such compression methods comes with several issues:
low compression ratio because the redundancy of the data is not fully used
slow compression and decompression

There also exists compression methods specialized in FASTQ encoding, divided in reference or non reference-based methods. However, none of them are fully satisfying, since a) the reference-based methods have good compression ratios but are slow, b) the non reference-based methods are faster but have lower compression ratios. An example of such a non reference-based method is provided by the software SPRING, which is a reference-free compressor for FASTQ files (worldwide web address: github.com/shubhamchandak94/SPRING). However, the compression method provided by the software SPRING has a low compression ratio.

Among the reference-based compression methods, some methods that use sequence alignments and are aimed to be faster with good compression ratios have been proposed. However, such methods suffer from several problems, notably a major issue is that they are not completely lossless. Such a known reference-based compression method is for example described in the patent document WO 2018/068829 A1. In the described method, after having been aligned to one or more reference sequences, the sequences of nucleotides are classified according to matching accuracy degrees (thereby creating classes of aligned reads), and are then coded as a multiplicity of layers of syntax elements, using different source models and entropy coders for each layer in which the data is partitioned. The classes of data are thus encoded separately and are structured in different layers of syntax elements, each layer comprising descriptors which univocally represent the classified and aligned reads of said layer. The method is intended to obtain distinct information sources with reduced information entropy, thereby allowing an increase in compression performance as well as a selective access to specific classes of compressed data. However, such a compression method reorders the reads in an order that is different from that obtained at the end of the read alignment step (i.e. the reads are reordered according to their classes). Some information is then lost in the compression process, notably the initial sequence ordering. Hence the reproducibility of some analysis results can be affected, because some downstream analysis software can be dependent on the order of the reads. Besides, decompressing the data in an order that is different from the initial order of the reads makes it much more difficult to check that the uncompressed file is identical to the initial file. Furthermore, such a compression method is relatively slow, especially when compared to the non reference-based compression methods of the state of the art.

### SUMMARY

The features of the independent claims below solve the problem of existing prior art solutions by providing a method for the compression of genome sequence data. In one aspect, a computer-implemented method for the compression of genome sequence data produced by a sequencing machine, said genome sequence data comprising reads of sequences of nucleotides or bases that have been aligned to a reference sequence, thereby creating aligned reads, said aligned reads being stored as a list of reads in an initial file, comprises the steps of:
for each aligned read, determining whether said read is perfectly or imperfectly mapped with said reference sequence or whether said read is unmapped with said reference sequence,
encoding the reads according to said determination, wherein the reads that are determined to be perfectly mapped are encoded according to a first encoding process and the reads that are determined to be unmapped are encoded according to a second encoding process,
wherein the determining step comprises comparing, for each imperfectly mapped read, the number of mismatches between said read and said reference sequence with a threshold value,
wherein, in the encoding step, the reads that are determined to be imperfectly mapped are encoded according to the second encoding process or to a third encoding process, the imperfectly mapped reads being encoded according to the second encoding process when said number of mismatches is greater than the threshold value, the imperfectly mapped reads being encoded according to the third encoding process when said number of mismatches is lower than the threshold value,
wherein, in said second encoding process, each nucleotide or base of the read is individually encoded,
wherein said first and third encoding processes comprise distinct sets of descriptors, each set of descriptors univocally representing the reads associated to the corresponding encoding process, each of said first and third encoding processes being a reduced information source entropy encoding process.

The invention overcomes the disadvantages of prior compression methods by allowing fast compression and decompression while causing no loss of information, and providing a high compression ratio. More particularly, the invention focuses on encoding the most frequent cases in the most compact way, even if this means adopting degraded encoding modes for the rare least frequent cases. This leads to a huge increase in compression performance. Moreover, due to the genomic information representation format that is used in the invention, the compression performed by the method according to the invention is faster. Last but not least, the method according to the invention keeps the initial order of the reads as such and does not reorder the reads according to their classes. Consequently, no information is lost during the process, which enables an easier downstream analysis as well as efficient conformity checks after the decompression step.

These and other features and advantages of the present invention will become more apparent from the accompanying drawings and the following detailed description.

### BRIEF DESCRIPTION OF THE DRAWINGS

FIG. 1 is a is a flow diagram showing the steps of the compression method according to the invention.

FIG. 2 is a diagram showing an apparatus for implementing the steps of the compression method according to the invention.

FIG. 3 shows a first example of a read that is globally mapped with a reference sequence.

FIG. 4 shows a second example of a read that is globally mapped with a reference sequence, in a case where a fake mismatch has to be inserted.

### DETAILED DESCRIPTION

The genomic sequences referred to in this invention include, for example, and not as a limitation, nucleotide sequences, Deoxyribonucleic acid (DNA) sequences, Ribonucleic acid (RNA), and amino acid sequences. Although the description herein is in considerable detail with respect to genomic information in the form of a nucleotide sequence, it will be understood that the compression method according to the invention can be implemented for other genomic sequences as well, albeit with a few variations, as will be understood by a person skilled in the art.

Genome sequencing information is generated by sequencing machines in the form of sequences of nucleotides (or, more generally, bases) represented by strings of letters from a defined vocabulary. The smallest vocabulary is represented by five symbols: {A, C, G, T, N} representing the 4 types of nucleotides present in DNA namely Adenine, Cytosine, Guanine, and Thymine. In RNA Thymine is replaced by Uracil (U). N indicates that the sequencing machine was not able to call any base and so the real nature of the position is undetermined.

The nucleotide sequences produced by sequencing machines are called “reads”. Sequence reads can be between a few dozens to several thousand nucleotides long. Some technologies produce sequence reads in pairs where one read is from one DNA strand and the second is from the other strand. Throughout this disclosure, a “reference sequence” is any sequence on which the nucleotide or base sequences produced by sequencing machines are aligned/mapped. One example of such a reference sequence could actually be a reference genome, i.e. a sequence assembled by scientists as a representative example of a species' set of genes. However, a reference sequence could also consist of a synthetic sequence conceived to merely improve the compressibility of the reads in view of their further processing. Sequencing machines can introduce errors in the sequence reads, and notably a use of a wrong symbol (i.e. representing a different nucleic acid) to represent the nucleic acid or base actually present in the sequenced sample; this is usually called a substitution error or a “mismatch”.

The invention is a reference-based compression method that receives reads of sequences of nucleotides or bases as inputs, such reads having been previously aligned to a reference sequence, thereby creating aligned reads. The aligned reads are then stored as a list of reads in an initial file. The way to align reads and to store them once aligned in an initial file is not critical to the invention and is not the purpose of the present disclosure. Each read is then encoded as a position on the reference sequence and a list of differences with said reference sequence. Each read can then be reconstructed from the alignment encoded information and the reference sequence, by a proper decompression software configured according to the present invention.

Preferably, the alignment software which processes the reads and aligns them to the reference sequence prior to providing them as inputs to the compression software and apparatus does not take into account certain types of errors introduced in the sequence reads, such as for example insertion errors or deletion errors. An insertion error consists in the insertion in one sequence read of one ore more additional symbols that no not refer to any actually present nucleic acid. A deletion error consists in the deletion from one sequence read of one or more symbols that represent nucleic acids that are actually present in the sequenced sample. More precisely, in case of an insertion error or a deletion error in a given sequence read, the alignment software will then consider the resulting erroneous nucleic acids as substitution errors, also called “mismatches”. This preferential choice for the alignment software configuration allows faster subsequent coding, providing notably a better compromise between speed and compression ratio.

For each read, the alignment software provides a corresponding read record to the compression software and apparatus. Each read record contains at least the following information: the absolute starting position of the aligned read with respect to the reference sequence, the length of the read, the type of alignment of the read, the number of mismatches identified in the read, and the relative position of said possible mismatches in the read (where appropriate).

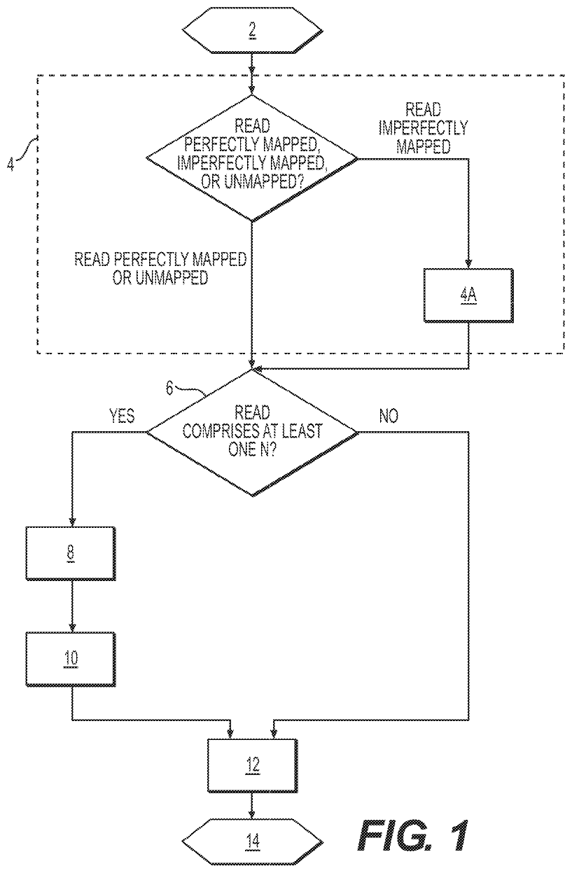

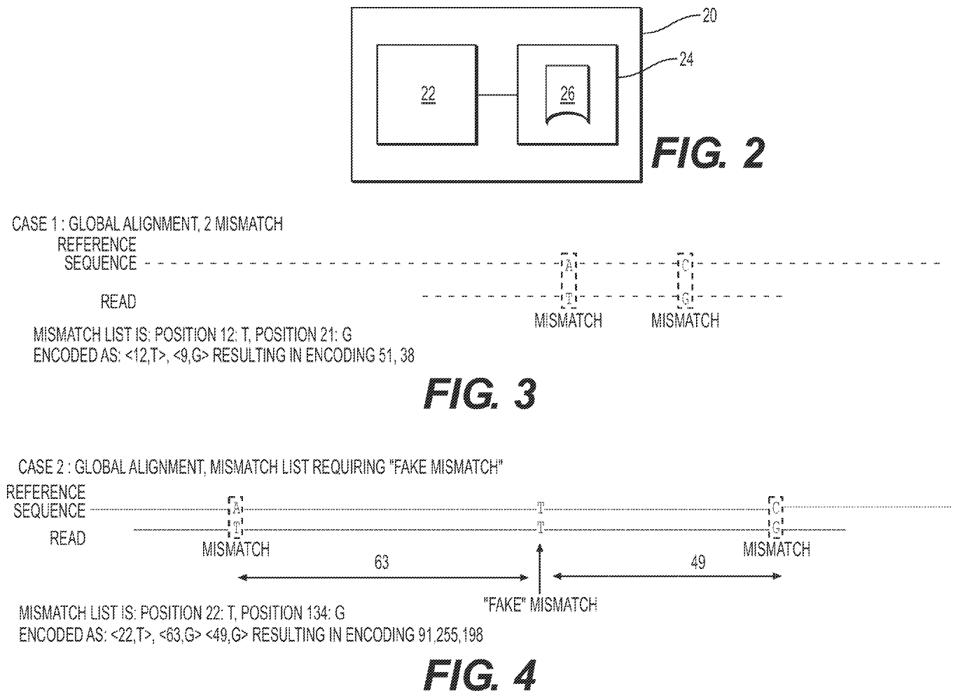

The compression method according to the present invention will now be described with reference to FIG. 1. The method is for example performed by an apparatus 20 shown in FIG. 2. The apparatus comprises at least one processor 22 and one memory 24 operatively coupled to the processor 22 to form a computing device. The memory 24 may store a computer program code or software 26 comprising computer executable instructions which, when executed by the processor 22, cause the processor 22 to perform operations comprising the steps of the compression method according to the invention.

The initial file in which the aligned reads are stored as a list of reads is for example stored in a memory of the apparatus 20. Returning to FIG. 1, the method preferably comprises an initial step 2 wherein the initial list of aligned reads is divided into blocks of reads. Typically, the list of aligned reads is divided into blocks of 50 000 reads, this specific value not being construed as limiting the scope of the present invention which can be applied in the same way with other values. Preferably, the blocks of reads have the same block size. Each block of reads begins with a header containing information needed to decode the block, such as for example the size in bytes of the content of the block, and/or an identifier of the block or its content and/or the number of reads contained in the block. This allows support for the concatenation of compressed file, as well as streaming capabilities (each block of reads containing all the information needed to decode the reads of the block). Besides, since the compression method can then be performed block after block, this also allows multi thread processing on the blocks of reads, thereby allowing parallelization and some resulting gain in processing time. If all the reads of a given block have the same length, the read length is also stored in the header, otherwise a list of each read length is stored explicitly during the compression method.

Each read record contains information about the type of alignment of the read. Typically, two main types of alignment can be identified: perfect alignment and imperfect alignment, plus an additional type corresponding to an “unmapped” read. “Imperfect alignment” means that the read contains at least one mismatch other than a N, while at least a portion of the read matches a portion of the reference sequence (according to this definition, an imperfectly mapped read may contain one or more N, provided it also contains one or more other mismatches). In an exemplary embodiment, each read record starts with the following bit flags, each bit flag having one value among two possible values:
a first bit flag indicative of a forward or reverse orientation versus the reference sequence,
a second bit flag indicative of a perfect alignment or not,
a third bit flag indicative of whether the read contains at least one N,
a fourth bit flag indicative of whether the position information is encoded on 16 bits or 32 bits.

The following steps 4-12 are performed block of reads after block of reads, and read after read within the blocks.

The method comprises a next step 4 of determining, for each aligned read, whether said read is perfectly or imperfectly mapped with the reference sequence, or whether said read is unmapped with the reference sequence. This determining step 4 comprises, for each imperfectly mapped read, comparing 4a the number of mismatches between said read and the reference sequence with a threshold value. In a preferred embodiment, though not to be construed as limiting the scope of the present invention, said threshold value is equal to 31. This specific value has been purposely chosen so as to provide the best possible compromise for storing the number of mismatches in a sufficiently compact manner, as will be better understood later with regard to step 12. Indeed, it has been statistically observed that in a vast majority of the cases, the imperfectly mapped reads have less than 31 mismatches. The principle lying behind that choice consists in encoding in the most compact way the most frequent cases, leave to have some very few degraded cases. If a read is determined to be imperfectly mapped with a number of mismatches lower than the threshold value, the determining step 4 comprises a further determination as to whether the read is globally or locally mapped with the reference sequence. A “globally mapped read” is an imperfectly mapped read whose whole sequence, comprising the beginning and the end of the read, is imperfectly mapped with the reference sequence. A “locally mapped read” is an imperfectly mapped read containing a segment of nucleotides or bases that is imperfectly mapped with the reference sequence. Said segment of nucleotides or bases thus corresponds to a portion of the initial read.

Preferably, the method further comprises a step 6 of determining, for each aligned read, whether said read comprises at least one N, i.e. whether said read comprises at least one mismatch corresponding to a case in which the sequencing machine was not able to call any base or nucleotide. The method then comprises, for each read comprising at least one N, a step 8 of determining the number of such N mismatches and a step 10 of comparing said number of N mismatches with a reference threshold value. In a preferred embodiment, though not to be construed as limiting the scope of the present invention, said reference threshold value is equal to 31.

Whatever the outcome of the determination step 4, the method comprises a next step 12 of encoding the reads according to said determination at least. More precisely, the reads that are determined to be perfectly mapped with the reference sequence, whether they comprise no N or has a number of N lower than the reference threshold value, are encoded according to a first encoding process. The reads that are determined to be unmapped or the reads that are determined to be perfectly mapped but with a number of N greater than the reference threshold value are encoded according to a second encoding process in which each nucleotide or base is individually encoded, regardless of whether said nucleotide or base is aligned or not. The reads that are determined to be imperfectly mapped are encoded according to the second encoding process or to a third encoding process. More precisely, the reads that are determined to be imperfectly mapped with a number of mismatches greater than the threshold value are encoded according to the second encoding process. If a read is determined to be imperfectly mapped with a number of mismatches lower than the threshold value, if said read comprises no N or has a number of N lower than the reference threshold value, then said read is encoded according to the third encoding process. If not, i.e. if the read has a number of N greater than the reference threshold value, then said read is encoded according to the second encoding process.

Whether a given read has been determined as being perfectly mapped, imperfectly mapped or unmapped, if said read comprises at least one N but has a number of N lower than the reference threshold value, the encoding step 12 comprises encoding a list of positions along the reference sequence, said positions corresponding to the positions of the N in the reference sequence. The list of positions is then stored in a memory of a computing device, said device implementing the compression method. If a read comprises at least one N but has a number of N lower than the reference threshold value, and is to be encoded according to the second encoding process, then each nucleotide or base of the read is individually encoded on 2 bits.

If a read comprises at least one N but with a number of N greater than the reference threshold value, then said read is in any case encoded according to the second encoding process, and each nucleotide or base of the read is individually encoded on 4 bits. In this case, the encoding step 12 does not comprise encoding and storing a list of positions of the N in the reference sequence. Indeed, each N mismatch is then directly encoded according to the second encoding process, in the very same way as the other nucleotides or bases of the read.

The first and third encoding processes comprise distinct sets of descriptors. Each set of descriptors univocally represents the reads associated to the corresponding encoding process, each of the first and third encoding processes being a reduced information entropy encoding process. More precisely, the third encoding process comprises a first encoding subprocess and a second encoding subprocess. The imperfectly mapped reads that are determined to be globally mapped during step 4 are encoded according to the first encoding subprocess. The imperfectly mapped reads that are determined to be locally mapped during step 4 are encoded according to the second encoding subprocess. The first and second encoding subprocesses comprise distinct sets of descriptors, each set of descriptors univocally representing the reads associated to the corresponding encoding subprocess.

The alignment information encoded for each read, and which enables the reconstruction of the whole read sequence during the decompression of the data, then depends on the corresponding encoding process or subprocess used for said read. For example, the descriptors used for the first encoding process may be:
the absolute starting position of the perfectly mapped read with respect to the reference sequence (encoded on 16 or 32 bits), and
the length of the read (encoded with differential coding relative to the length of the previous read, with variable length code ranging from 2 bits to 34 bits).

The descriptors used for the first encoding subprocess may be:
the absolute starting position of the imperfectly mapped read with respect to the reference sequence (encoded on 16 or 32 bits),
the length of the read (encoded with differential coding relative to the length of the previous read, with variable length code ranging from 2 bits to 34 bits), and
a list of the mismatches of the read.

The descriptors used for the second encoding subprocess may be:
the absolute starting position of the imperfectly mapped portion of the read with respect to the reference sequence—also called local alignment starting position (encoded on 16 or 32 bits),
the length of the read (encoded with differential coding relative to the length of the previous read, with variable length code ranging from 2 bits to 34 bits),
a list of the mismatches of the read, and
the length of the clipped portions of the read that are not part of the alignment (encoded on 8 bits for each clipped portion).

Preferably, the list of mismatches which is encoded in the first and second subprocesses comprises a header (bit flag, encoded on 1 byte). The five first bits of the byte are used to encode the number of mismatches contained in the read (in the preferred embodiment wherein the threshold value is equal to 31, said number is within the range [0-31]). One bit is then used to encode whether the imperfectly mapped read is globally or locally mapped. Another bit is used to encode whether or not the 2-bit mode is activated for the second encoding process. The last bit is used to encode whether or not the 4-bit mode is activated for the second encoding process. Preferably, for each read encoded according to the second encoding subprocess during the encoding step 12, the clipped portions of said read (i.e. those portions that are not part of the local alignment) are concatenated, and each nucleotide or base of said clipped portions is individually encoded. In a preferred embodiment, each nucleotide or base of such clipped portions of the read is individually encoded on 2 bits.

In a preferred embodiment, each mismatch encoded in the list of mismatches of an imperfectly mapped read (i.e. encoded according to the first or second encoding subprocess) is encoded on 1 byte. More precisely, each mismatch of an imperfectly mapped read that is to be encoded according to the first or second encoding subprocess may be encoded as follows:
the two first bits of the byte are used to encode the alternate nucleotide or base present in the read instead of the corresponding reference nucleotide or base in the reference sequence,
the six last bits are used to encode the position of the mismatch in the reference sequence, said position being computed as an offset from the previous mismatch of the read (relative position of the mismatch, except for the first mismatch of the read for which the absolute position is encoded). The range of this offset, which is encoded on 6 bits, is therefore [0-63].

FIG. 3 provides an example of the encoding of the mismatches of a read according to the first encoding subprocess. The read is an imperfectly mapped read, which is globally mapped with the reference sequence. The read has two mismatches:
a first mismatch, located in the 12^{th} position in the read, which consists in a substitution of a A nucleotide in the reference sequence by a T nucleotide in the read, and
a second mismatch, located in the 21^{th} position in the read, which consists in a substitution of a C nucleotide in the reference sequence by a G nucleotide in the read.

The list of the mismatches of the read is then encoded as:
<12, T>, the value “12” corresponding to the absolute position of the first mismatch in the read, and
<9, G>, the value “9” corresponding to the relative position of the second mismatch in the read, i.e. the offset between the second mismatch and the first mismatch.

<12, T> may for example be converted into the value “51” (encoded on 1 byte), and <9, G> may be converted into the value “38” (encoded on 1 byte). Such a byte encoding is obtained with:

offset position×4+nucleotide value(with A=0,C=1,G=2,T=3)

Preferably, for each imperfectly mapped read that is to be encoded according to the first or second encoding subprocess, if the offset computed between a given mismatch of the read and the previous mismatch is greater than a maximum encodable value, then at least one “fake” mismatch is inserted between said two mismatches until every offset between each of said mismatches and the at least one “fake” mismatch is lower than said maximum encodable value. A “fake” mismatch is defined as a mismatch for which the bits of the byte used to encode the mismatch encode a nucleotide or base that is equal to the corresponding reference nucleotide or base in the reference sequence. In a preferred embodiment, though not to be construed as limiting the scope of the present invention, the maximum encodable value is equal to 63, corresponding to the maximum value that is encodable on 6 bits.

FIG. 4 provides an example of the encoding of the mismatches of a read according to the first encoding subprocess, in a case where a “fake” mismatch has to be inserted. The read is an imperfectly mapped read, which is globally mapped with the reference sequence. The read has two mismatches:
a first mismatch, located in the 22^{th} position in the read, which consists in a substitution of a A nucleotide in the reference sequence by a T nucleotide in the read, and
a second mismatch, located in the 134^{th} position in the read, which consists in a substitution of a C nucleotide in the reference sequence by a G nucleotide in the read.

The position offset between the second and the first mismatches is of 112, which is greater than the maximum encodable value of 63. A “fake” mismatch therefore has to be inserted between the two mismatches, so that every offset between each of the mismatches and the “fake” mismatch is lower than said maximum encodable value. A “fake” mismatch with a T nucleotide (corresponding to a “real” T nucleotide in the reference sequence) is for example inserted in the 85^{th} position in the read. The position offset computed between the “fake” mismatch and the first mismatch is 63, which is corresponds to the maximum encodable value. The position offset computed between the second mismatch and the “fake” mismatch is of 49, which is lower than 63.

The list of the mismatches of the read is then encoded as:
<22, T>, the value “22” corresponding to the absolute position of the first mismatch in the read,
<63, T>, the value “63” corresponding to the relative position of the “fake” mismatch in the read, i.e. the offset between the “fake” mismatch and the first mismatch, and
<49, G>, the value “49” corresponding to the relative position of the second mismatch in the read, i.e. the offset between the second mismatch and the “fake” mismatch.

<22, T> may for example be converted into the value “91” (encoded on 1 byte), <63, T> may be converted into the value “255” (encoded on 1 byte), and <49, G> may be converted into the value “198” (encoded on 1 byte). Such a byte encoding is obtained with:

offset position×4+nucleotide value(with A=0,C=1,G=2,T=3)

The method comprises a final step 14 of providing a compressed file comprising a list of encoded reads. The encoded reads are stored in the compressed file in the same order as that of the reads stored in the initial uncompressed file. Each read can then be reconstructed from the alignment encoded information and the reference sequence, by a proper decompression software and/or method configured according to the present invention.

Although described with reference to an exemplary architecture of a computing device 20 (shown in FIG. 2 for illustrative purposes), the inventive techniques herewith disclosed may be implemented in hardware, software, firmware or any combination thereof. When implemented in software, the computer program code may be stored on a computer medium and executed by a hardware processing unit comprising one or more processors, as is the case with the device 20 of FIG. 2. It should be understood that the term “processor” as used herein is intended to include one or more processing devices, including a signal processor, a microprocessor, a microcontroller, an application-specific integrated circuit (ASIC), a field programmable gate array (FPGA) or other type of processing circuitry, as well as portions or combinations of such circuitry elements. Also, the term “memory” as used herein is intended to include electronic memory associated with a processor, such as random access memory (RAM), read-only memory (ROM) or other types of memory, in any combination.

Accordingly, software instructions or code for performing the methodologies and protocols described herein may be stored in one or more of the associated memory devices, e.g., ROM, fixed or removable memory, and, when ready to be utilized, loaded into RAM and executed by the processor.

The techniques of this disclosure may be implemented in a wide variety of devices or apparatuses, including for example mobile phones, computers, servers, tablets and similar devices.

Although illustrative embodiments of the present invention have been described herein with reference to the accompanying drawings, it is to be understood that the invention is not limited to those precise embodiments, and that various other changes and modifications may be made by one skilled in the art without departing from the scope or spirit of the invention.

### Statistical and Numerical Examples of the Compression Method According to the Invention

The following comparative example has been performed on an uncompressed data file that contained 48 million reads or sequences of nucleotides:
size of the uncompressed data file: 35,770 MB (MegaByte)
size of the file that has been compressed with the gzip software: 6,649 MB
size of the file that has been compressed with the non reference-based SPRING software: 1,402 MB
size of the file that has been compressed with the reference-based compression method according to the present invention: 1,179 MB
compression time with the non reference-based SPRING software: 1,722 s
compression time with the reference based-compression method according to the present invention: 181 s
average size in Bit/Nucleotide of the uncompressed data file (ASCII encoding): 8 bit/nucleotide
average size in Bit/Nucleotide of the file that has been compressed with a coding adapted to 4 possible characters A, T, C, G: 2 bit/nucleotide
average size in Bit/Nucleotide of the file that has been compressed with the reference-based compression method according to the present invention: 0.33 bit/nucleotide

The numerical examples indicated above illustrate that the present invention allows for fast compression and decompression, while providing a high compression ratio.

1-17. (canceled)

18. A method for compressing genomic sequence data, the method comprising:
obtaining, by one or more computers, a read record;
determining, by the one or more computers, whether the read record corresponds to a read that is perfectly mapped to a reference sequence or imperfectly mapped to the reference sequence;
based on determining, by the one or more computers, that the read record corresponds to a read that is imperfectly mapped to the reference sequence, determining, by the one or more computers, whether a number of mismatches of the imperfectly mapped read satisfies a predetermined threshold number of mismatches; and
based on determining that the number of mismatches satisfies the predetermined threshold number of mismatches, (i) obtaining, by the one or more computers, an offset from a previous mismatch that is lower than a maximum encodable offset value and (ii) encoding, by the one or more computers, each mismatch of the imperfectly mapped read and the offset from the previous mismatch of the read into a record having a size of 1 byte.

19. The method of claim 18, wherein determining, by the one or more computers, whether a number of mismatches of the imperfectly mapped read satisfies a predetermined threshold number of mismatches comprises:
determining, by the one or more computers, whether the number of mismatches of the imperfectly mapped read is greater than the predetermined threshold number of mismatches.

20. The method of claim 18, wherein each read record comprises:
data indicating an absolute starting position of an aligned read with respect to the reference sequence;
data indicating a length of the read;
data indicating whether the read is perfectly mapped or imperfectly mapped;
data indicating a number of mismatches identified in the read; and
data indicating a relative position of each of said mismatches in the read.

21. The method of claim 18, wherein encoding each mismatch of the imperfectly mapped read into a record having a size of 1 byte comprises for each particular mismatch:
encoding, by the one or more computers, a first two bits of the byte to include data representing an alternate nucleotide or base present in the read instead of a corresponding reference nucleotide or base in the reference sequence; and
encoding, by one or more computers, six remaining bits of the byte to include data representing the offset.

22. The method of claim 21, the method further comprising:
determining, by one or more computers, whether the offset is greater than a maximum encodable value; and
based on determining that the offset is greater than the maximum encodable value, inserting, by one or more computers, at least one fake mismatch between the particular mismatch and the previous mismatch.

23. The method of claim 18, the method further comprising:
based on determining that the number of mismatches does not satisfy the predetermined threshold number of mismatches, encoding, by one or more computers, a list of positions of the reference sequence corresponding to a position of each of the mismatches to the reference sequence using a reduced information entropy encoding process.

24. The method of claim 18, the method further comprising:
based on determining that the read record corresponds to a read that is perfectly mapped to the reference sequence, encoding, by one or more computers, at least a portion of the read record using reduced information entropy encoding.

25. A hardware processor that includes hardware processing circuitry that is configured to perform one or more operations, the one or more operations comprising:
obtaining, by the hardware processing circuitry, a read record;
determining, by the hardware processing circuitry, whether the read record corresponds to a read that is perfectly mapped to a reference sequence or imperfectly mapped to the reference sequence;
based on determining, by the hardware processing circuitry, that the read record corresponds to a read that is imperfectly mapped to the reference sequence, determining, by the one or more computers, whether a number of mismatches of the imperfectly mapped read satisfies a predetermined threshold number of mismatches; and
based on determining that the number of mismatches satisfies the predetermined threshold number of mismatches, (i) obtaining, by the hardware processing circuitry, an offset from a previous mismatch that is lower than a maximum encodable offset value and (ii) encoding, by the hardware processing circuitry, each mismatch of the imperfectly mapped read and the offset from the previous mismatch of the read into a record having a size of 1 byte.

26. The hardware processor of claim 25, wherein each read record comprises:
data indicating an absolute starting position of the aligned read with respect to the reference sequence;
data indicating a length of the read;
data indicating whether the read is perfectly mapped or imperfectly mapped;
data indicating a number of mismatches identified in the read; and
data indicating a relative position of said mismatches in the read.

27. The hardware processor of claim 25, wherein encoding each mismatch of the imperfectly mapped read into a record having a size of 1 byte comprises for each particular mismatch:
encoding, by the hardware processing circuitry, a first two bits of the byte to include data representing an alternate nucleotide or base present in the read instead of a corresponding reference nucleotide or base in the reference sequence; and
encoding, by the hardware processing circuitry, a six remaining bits of the byte to include data representing the offset.

28. The hardware processor of claim 27, wherein the hardware processor circuitry is further configured to perform operations comprising:
determining, by the hardware processing circuitry, whether the offset is greater than a maximum encodable value;
based on determining that the offset is greater than the maximum encodable value, inserting, by the hardware processing circuitry, at least one fake mismatch between the particular mismatch and the previous mismatch.

29. The hardware processor of claim 25, wherein the hardware processor circuitry is further configured to perform operations comprising:
based on determining that the number of mismatches does not satisfy the predetermined threshold number of mismatches, encoding, by the hardware processing circuitry, a list of positions of the reference sequence corresponding to a position of each of the mismatches to the reference sequence using a reduced information entropy encoding process.

30. The hardware processor of claim 25, wherein the hardware processor circuitry is further configured to perform operations comprising:
based on determining that the read record corresponds to a read that is perfectly mapped to the reference sequence, encoding, by the hardware processing circuitry, at least a portion of the read record using reduced information entropy encoding.

31. The hardware processor of claim 25 wherein the hardware processing circuitry comprises one or more field programmable gate arrays (FPGAs).

32. The hardware processor of claim 25, wherein determining, by the hardware processing circuitry, whether a number of mismatches of the imperfectly mapped read satisfies a predetermined threshold number of mismatches comprises:
determining, by the hardware processing circuitry, whether the number of mismatches of the imperfectly mapped read is greater than the predetermined threshold number of mismatches.

33. A system for compressing genomic sequence data, the system comprising:
one or more computers and one or more storage devices storing instructions that are operable, when executed by one or more computers, to cause the one or more computers to perform the operations comprising:
obtaining, by the one or more computers, a read record;
determining, by the one or more computers, whether the read record corresponds to a read that is perfectly mapped to a reference sequence or imperfectly mapped to the reference sequence;
based on determining, by the one or more computers, that the read record corresponds to a read that is imperfectly mapped to the reference sequence, determining, by the one or more computers, whether a number of mismatches of the imperfectly mapped read satisfies a predetermined threshold number of mismatches; and
based on determining that the number of mismatches satisfies the predetermined threshold number of mismatches, (i) obtaining, by the one or more computers, an offset from a previous mismatch that is lower than a maximum encodable offset value and (ii) encoding, by the one or more computers, each mismatch of the imperfectly mapped read and the offset from the previous mismatch of the read into a record having a size of 1 byte.

34. The system of claim 33, wherein each read record comprises:
data indicating an absolute starting position of an aligned read with respect to the reference sequence;
data indicating a length of the read;
data indicating whether the read is perfectly mapped or imperfectly mapped;
data indicating a number of mismatches identified in the read; and
data indicating a relative position of each of said mismatches in the read.

35. The system of claim 33, wherein encoding each mismatch of the imperfectly mapped read into a record having a size of 1 byte comprises for each particular mismatch:
encoding, by one or more computers, a first two bits of the byte to include data representing an alternate nucleotide or base present in the read instead of a corresponding reference nucleotide or base in the reference sequence; and
encoding, by one or more computers, six remaining bits of the byte to include data representing the offset.

36. The system of claim 35, the operations further comprising:
determining, by the one or more computers, whether the offset is greater than a maximum encodable value; and
based on determining that the offset is greater than the maximum encodable value, inserting, by one or more computers, at least one fake mismatch between the particular mismatch and the previous mismatch.

37. The system of claim 33, the operations further comprising:
based on determining that the number of mismatches does not satisfy the predetermined threshold number of mismatches, encoding, by one or more computers, a list of positions of the reference sequence corresponding to a position of each of the mismatches to the reference sequence using a reduced information entropy encoding process.

38. The system of claim 33, the operations further comprising:
based on determining that the read record corresponds to a read that is perfectly mapped to the reference sequence, encoding, by one or more computers, at least a portion of the read record using reduced information entropy encoding.

39. The system of claim 33, wherein the one or more computers comprises one or more hardware processors.

40. The system of claim 39 wherein the one or more hardware processors comprises one or more field programmable gate arrays (FPGAs).

41. A computer-readable storage device having stored thereon instructions, which, when executed by a data processing apparatus, cause the data processing apparatus to perform operations for compressing genomic sequence data, the operations comprising:
obtaining a read record;
determining whether the read record corresponds to a read that is perfectly mapped to a reference sequence or imperfectly mapped to the reference sequence;
based on determining that the read record corresponds to a read that is imperfectly mapped to the reference sequence, determining whether a number of mismatches of the imperfectly mapped read satisfies a predetermined threshold number of mismatches; and
based on determining that the number of mismatches satisfies the predetermined threshold number of mismatches, (i) obtaining an offset from a previous mismatch that is lower than a maximum encodable offset value and (ii) encoding each mismatch of the imperfectly mapped read and the offset from the previous mismatch of the read into a record having a size of 1 byte.

42. The computer-readable storage device of claim 41, wherein each read record comprises:
data indicating an absolute starting position of an aligned read with respect to the reference sequence;
data indicating a length of the read;
data indicating whether the read is perfectly mapped or imperfectly mapped;
data indicating a number of mismatches identified in the read; and
data indicating a relative position of each of said mismatches in the read.

43. The computer-readable storage device of claim 41, wherein encoding each mismatch of the imperfectly mapped read into a record having a size of 1 byte comprises for each particular mismatch:
encoding, by one or more computers, a first two bits of the byte to include data representing an alternate nucleotide or base present in the read instead of a corresponding reference nucleotide or base in the reference sequence; and
encoding, by one or more computers, six remaining bits of the byte to include data representing the offset.

44. The computer-readable storage device of claim 43, the operations further comprising:
determining, by the one or more computers, whether the offset is greater than a maximum encodable value; and
based on determining that the offset is greater than the maximum encodable value, inserting, by one or more computers, at least one fake mismatch between the particular mismatch and the previous mismatch.

45. The computer-readable storage device of claim 41, the operations further comprising:
based on determining that the number of mismatches does not satisfy the predetermined threshold number of mismatches, encoding a list of positions of the reference sequence corresponding to a position of each of the mismatches to the reference sequence using a reduced information entropy encoding process.

46. The computer-readable storage device of claim 41, the operations further comprising:
based on determining that the read record corresponds to a read that is perfectly mapped to the reference sequence, encoding at least a portion of the read record using reduced information entropy encoding.
